(19) United States
(12) Patent Application Publication
(10) Pub. No.: US 2019/0360922 A1
(43) Pub. Date: Nov. 28, 2019

(54) DETERMINATION OF STARCH IN A SAMPLE AT AN INDUSTRIAL FACILITY

(71) Applicant: BUCKMAN LABORATORIES, Memphis, TN (US)

(72) Inventors: Jian TAN, Bartlett, TN (US); Rosa COVARRUBIAS, Memphis, TN (US); Hannes MONSTREY, Eke (BE)

(21) Appl. No.: 16/414,875

(22) Filed: May 17, 2019

Related U.S. Application Data
(60) Provisional application No. 62/674,889, filed on May 22, 2018.

(51) Int. Cl. G01N 21/27; G01N 21/59; G01N 21/78; G01N 33/34

(52) U.S. Cl. CPC G01N 21/272; G01N 21/59; G01N 2021/7783; G01N 33/343; G01N 21/78

(57) ABSTRACT

In a starch concentration measurement, a liquid sample is conducted from a liquid sample such as pulp suspension or filtrate of a paper, board or tissue process. An iodine solution is added to the sample, and a light absorbance or transmittance of the sample is measured at a target wavelength. The measured absorbance or transmittance of the sample is then converted into the starch concentration of the sample.

### CROSS-REFERENCE TO RELATED APPLICATIONS

This application is a Non-Provisional application, which claims priority to U.S. Provisional Application 62/674,889, filed 22 May 2018, hereby incorporated by reference in its entirety.

### FIELD OF THE INVENTION

The invention relates to measurement technology of industrial liquids containing starch. In particular, the invention presents an on-site method of measuring starch concentration in an industrial facility such as a paper mill.

### BACKGROUND OF THE INVENTION

Starch is commonly used in paper, e.g., to increase paper strength. However, soluble starch is an interfering substance that can cause severe runnability and microbial problems, and papermakers should therefore try to minimize the starch concentration in the process waters. Starch which has been adsorbed onto fibers and other particles, i.e., an adsorbed starch, is not generally a problem for the papermaker but could be of interest.

Starch concentration is typically measured off-site (i.e., a sample is taken from an industrial facility, such as a paper mill, and sent to a laboratory for testing) and can, for example, be analyzed by gas chromatography after hydrolysis into monomeric glucose. However, this is a very time-consuming process. It is desired to provide a method for performing this task on-site to avoid the disadvantages of off-site testing.

Due to the length of time consumed by and complexity of off-site analysis of starch concentration, there exists a need for fast, reliable, and on-site analysis methods for determining starch concentrations of industrial liquids.

### SUMMARY OF THE INVENTION

An aspect of the invention is a method of analyzing starch concentration in a liquid sample. An aspect of the invention is a method of analyzing starch concentration in a liquid sample, comprising
obtaining a sample from a stream of liquid;
adding iodine solution to the sample;
measuring a light absorbance or transmittance of the sample after the step of adding the iodine solution;
converting the measured absorbance or transmittance of the sample into the starch concentration of the sample by means of a predefined correlation between a starch concentration and a light absorbance or transmittance, wherein the light absorbance or transmittance is measured at a wavelength between 600-630 nm. In an embodiment, the light absorbance or transmittance is measured at a wavelength between 610-625 nm. In another embodiment, the light absorbance or transmittance is measured at a wavelength of 620 nm.

In an embodiment, the method comprises determining a ratio of dissolved and absorbed starch in the sample based on the starch concentrations of one or more particle populations.

In an embodiment, the one or more particle populations include one or more of colloids, fines, fibers, floccules and agglomerates.

In an embodiment, the liquid sample is conducted from a pulp suspension or filtrate in a paper, board or tissue process.

An aspect of the invention is a measurement system implementing the analyzing method.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the following, exemplary embodiments of the invention will be described with reference to the attached drawings, in which

FIG. 1A shows a table of measured absorbance values;

FIG. 1B shows a standard starch working curve (i.e., a graph of the average of the measured absorbance values of FIG. 1A);

FIG. 2 graphically represents the influence of starch on volatile fatty acids in a pre-acidification test; and

FIGS. 3-7 show, for various samples, percentage free starch in solution, percentage total starch in solution, and percentage of fiber-bonded starch out of total starch.

### DETAILED DESCRIPTION OF THE INVENTION

Traditional spectrophotometric determination of starch concentration in a water sample is typically done by adding a known amount of iodine in the sample and a reference cell or alternatively using a blank sample with water and iodine for base line correction. Traditionally the absorbance of iodine/starch complex is measured at a wavelength of 580 nm which is the overall maxima of common wet end starches.

The present invention uses a faster measurement of dissolved starch than the iodine starch method. The method is based on the reaction between iodine/potassium iodide and starch. Iodine/potassium iodide changes color when combined with amylose and amylopectin with absorption maxima at 605 nm (nanometers) and 530 nm, respectively. Traditionally the absorbance is measured at 580 nm, which is the overall maxima of common wet end starches.

The present inventors have discovered that wavelengths greater than 580 nm are useful for on-site determinations of starch concentration of samples industrial facilities. According to an aspect of the invention, a light absorbance or transmittance of the sample after adding the iodine solution may be measured at a wavelength of between 600-630 nm. In an embodiment, the light absorbance or transmittance is measured at a wavelength between 610-625 nm. In another embodiment, the light absorbance or transmittance is measured at a wavelength of 620 nm. However, the light absorption is to some extent dependent on the degree of modification of starch. Therefore, the method of the invention can be calibrated for different starches independently using a linear regression calibration between known amounts of starch and the absorbance or transmittance of light at a wavelength between 600-630 nm.

International Publication No. WO 2017/168045 A1 (“WO '045”) describes a method and system for determination of starch in a sample. In particular, WO '045 explains that the use of wavelengths longer than 650 nm are beneficial because WO '045 claims the calibration curve between absorption and starch concentration is not affected by the starch type or degree of modification of the starch. In contrast to WO '045, the present inventors have found that light absorbance or transmittance of the sample measured at a wavelength of between 600-630 nm is beneficial for on-site starch concentration determinations in industrial facilities.

In an embodiment, the light absorbance or transmittance is measured at a wavelength between 610-625 nm. In another embodiment, the light absorbance or transmittance is measured at a wavelength of 620 nm. Preferably, samples are both preacidified before being measured to provide more reliable measurements and pre-diluted to avoid turbidity concerns, if needed.

An exemplary embodiment of a method for measuring starch concentration in a liquid sample such as pulp suspension or filtrate in a paper, board (e.g., cardboard, containerboard) or tissue process is described as follows. A sample is provided from a liquid sample such as pulp suspension or filtrate of a paper, board or tissue process. In an embodiment, the sample is pre-diluted with distilled water to avoid turbidity concerns, if needed. An iodine solution is added to the sample. A light absorbance or transmittance of the sample is measured at a wavelength of 620 nm after adding the iodine solution. The measured absorbance or transmittance of the sample is converted into the starch concentration of the sample by means of a predefined correlation between a starch concentration and a light absorbance or transmittance. For example, a starch-standard working curve is prepared, as described below, and a formula for calculating starch concentration using measured absorbances or transmittances of the starch standards is then derived, as also described below. The derived formula is then used to calculate starch concentration based on measured absorbance or transmittance values of the sample.

In an embodiment, a first light absorbance or transmittance of the sample is measured at a wavelength between 600-630 nm. After the measurement, an iodine solution is added to the sample. A second light absorbance or transmittance of the sample is measured at a wavelength between 600-630 nm, preferably the same wavelength used to measure the first light absorbance or transmittance. A difference between the first and second measured absorbance or transmittance of the sample is converted into the starch concentration of the sample by means of a predefined correlation between a starch concentration and a light absorbance or transmittance.

The phrase “measurement at a wavelength” as used herein generally refers to a measurement at one wavelength with a spectral resolution set by a measurement arrangement in question, or refers to a measurement of a narrow spectra of wavelengths of, for example, 620 nm.

In an embodiment, a 0.01 M iodine (KI₃) solution is prepared by mixing 2.6 grams of potassium iodide (KI) and 0.13 grams of iodine (I2) and adding distilled water, and diluting to 100 g to make 0.01 M I₂/KI solution. Alternatively, instead of preparing the solution, commercially available iodine solutions may be used.

To prepare 6 M and 0.6 M HCl solutions, 37% concentrated HCl (12 M) is diluted in half to obtain 6 M HCl solution. 6 M HCl is then diluted by tenth to obtain 0.6 M HCl. Alternatively, instead of preparing the solution, commercially available solutions may be used.

To prepare standard starch solution, 6 100-mL beakers are used to prepare series dilution as shown below: 1%, 0.04%, 0.02%, 0.01%, 0.005%, and 0.0025%. The starch polymer BBD 616 is used as standard starch (25% solid content). See Table 1 below. Alternatively, a starch slurry may be used as a standard. Due to paper mills' variation in starch content (i.e., a mill may be a low- or high-starch content system), the standard curve should be adjusted accordingly. In an embodiment, starch used in a paper mill's machines is preferred over commercially available starch.

TABLE 1

| Target | Water | 25.000% | 1.000% | 0.040% | 0.020% | 0.010% | 0.005% |
|---|---|---|---|---|---|---|---|
| 1.0000% | 96.00 | 4.00 | | | | | |
| 0.0400% | 96.00 | | 4.00 | | | | |
| 0.0200% | 50.00 | | | 50.00 | | | |
| 0.0100% | 50.00 | | | | 50.00 | | |
| 0.0050% | 50.00 | | | | | 50.00 | |
| 0.0025% | 50.00 | | | | | | 50.00 |

0.02%, 0.01%, 0.005%, and 0.0025% solutions will be working solutions to generate starch concentration curve.

To prepare waste board pulp solution, a typical air-dry old corrugated container board (“OCC”) sample from waste board is collected and cut into small pieces with scissors and mixed well. 5.37 g of these small pieces are weighed and placed in a clean 1000-mL beaker and 494.63 g water are added to reach 500 g (small pieces and water). The oven-dry OCC weight is 5 g (5.37 g x 0.93=5.0 g). A handheld blender is used to disperse OCC flakes into 1%-consistency pulp slurry for 3 minutes. 50 g of 1.0% pulp slurry are measured and diluted with water to 500 g to obtain 0.1% pulp slurry.

To prepare headbox pulp slurry, collect headbox slurry and obtain consistency. If desired, dilute pulp slurry to 0.1%.

To prepare starch-standard working curve, 6 20-mL test tubes are labeled as “blank,” “0.0025%,” “0.0050%,” “0.010%,” “0.020%,” and “0.040%.” (Other concentrations, for example, 0.025%, may be used.) A 10-mL syringe is used to add 10 mL water and related starch solution to each test tube. Another 1-mL syringe is used to add 0.50 mL 0.6 M HCl to each test tube. Another 1-mL syringe is used to add 0.50 mL 0.01 M I₂/KI solution to the “blank” test tube. The “blank” test tube is then used to zero the spectrometer. Another 1-mL syringe is used to add 0.50 mL 0.01 M I₂/KI solution to the “0.0025%” test tube and placed in the spectrometer; the corresponding absorbance is then measured and recorded. Absorbance values for the 0.0050%, 0.010%, 0.020%, and 0.020% solutions are measured and recorded in the same manner. Coefficient a and R-squared values are then obtained. The test is repeated, dated are record data, and average absorbance values are calculated for each concentration.

To measure free starch in waste paperboard, filter paper and a glass funnel are used to filter 100 mL OCC 0.1% pulp slurry. A 10-mL syringe is used to obtain 10-mL of the filtrate, which is added to a test tube, followed by the addition of 0.5 mL 0.5 M HCL and 0.5 mL 0.01 M I₂/KI solution. In an embodiment, up to 1.0 mL 0.01 M I₂/KI solution may be added. Absorbance for each solution is measured and recorded, and the test is repeated to obtain average absorbance values for each solution. Starch concentration in solution=(average absorbance)/(coefficient a previously obtained from starch-standard working curve). Starch concentration in paperboard=1000*(starch concentration in solution).

To measure free starch in headbox slurry, 1000 mL headbox slurry are collected. 500 mL pulp slurry are used to make consistency pad to determine consistency x. As would be understood by one of skill in the art, a consistency pad, in this example, is a pad made with pulp to determine the dry weight of the 500 mL pulp slurry which determines discrete masses of dry pulp and water. Water is added to 0.1x grams slurry to make 100 mL 0.1% slurry. Filter paper and a glass funnel are used to filter 100 mL OCC 0.1% pulp slurry. A 10-mL syringe is used to obtain 10 mL filtrate, which is added to a test tube, followed by the addition of 0.5 mL 0.5 M HCl and 0.5 mL 0.01 M I₂/KI solution. In an embodiment, up to 1.0 mL 0.01 M I₂/KI solution may be added. Solution absorbance is recorded, the test is repeated, and an average absorbance is obtained. Starch concentration in 0.1% diluted slurry=(average absorbance)/(coefficient a previously obtained from starch-standard working curve). Starch concentration in headbox slurry=1000*(starch concentration in 0.1% diluted slurry)*(headbox pulp consistency). Starch relative to fiber weight=(starch concentration in headbox slurry)/(headbox pulp consistency).

To measure free starch in white water, 100 mL white water are collected. Filter paper and a glass funnel are used to filter the collected white water. A 10-mL syringe is used to obtain 10 mL filtrate, which is added to a test tube, followed by the addition of 0.5 mL 0.6 M HCl and 0.05 mL 0.01 M I₂/KI solution. In an embodiment, up to 1.0 mL 0.01 M I₂/KI solution may be added. Solution absorbance is recorded, the test is repeated, and an average absorbance is obtained. Starch concentration in white water=(average absorbance)/(coefficient a previously obtained from starch-standard working curve).

To measure overall starch (free starch and bonded starch) in waste paperboard, 100 grams of above 0.10% pulp slurry are added into a 200-mL beaker. 0.5 mL 6.0 M HCl is added into the beaker. A microwave oven or hot plate is used to bring solution to boil for about 1 minute. For thick stock, more time may be needed. (To determine if the solution was sufficiently boiled, after the sample is filtered, iodine solution may be dropped onto the fibers. If no purple color develops, there is no more starch attached to the fibers.) The solution is cooled to room temperature naturally or by water bath. Filter paper and a glass funnel are used to filter slurry and collect filtrate. A 10-mL syringe is used to obtain 10 mL filtrate, which is then added to a test tube, followed by the addition of 0.5 mL 0.01 M I₂/KI solution. In an embodiment, up to 1.0 mL 0.01 M I₂/KI solution may be added. Solution absorbance is recorded, the test is repeated, and an average absorbance is obtained. Overall starch concentration in solution=(average absorbance)/(coefficient a previously obtained from starch standard working curve). Overall starch concentration in board=1000*(overall starch concentration in solution). Bonded starch on fiber surface=(overall concentration in board)−(starch concentration in board).

To measure overall starch (free starch and bonded starch) in headbox slurry, 1000 mL headbox slurry are collected. 500 mL pulp slurry are used to make consistency pad to determine consistency. The slurry is diluted with water to make 100 mL 0.1% slurry. 100 g of 0.1% slurry are added into a 200-mL beaker. 0.5 mL 6.0 M HCl is added into beaker. A microwave oven or hot plate is used to let solution boil for 1 minute. The solution is cooled to room temperature naturally or by water bath. Filter paper and a glass funnel are used to filter 0.1% diluted headbox pulp slurry 100 mL. A 10-mL syringe is used to obtain 10 mL filtrate, which is then added to a test tube, followed by the addition of 0.5 mL 0.01 M I₂/KI solution. In an embodiment, up to 1.0 mL 0.01 M I₂/KI solution may be added. Solution absorbance is recorded, the test is repeated, and an average absorbance is obtained. Starch concentration in 0.1% diluted slurry=(average absorbance)/(coefficient a previously obtained from standard-starch working curve). Starch concentration in headbox slurry=(starch concentration in 0.1% diluted slurry)*(headbox pulp consistency)*(1000). Overall starch relative to fiber weight=(starch concentration in headbox slurry)/(headbox pulp consistency). Bonded starch on fiber surface=(overall starch relative to fiber weight)-(starch relative to fiber weight, previously obtained in measuring free starch in headbox slurry).

To show how amylase decomposes starch, two 20-mL test tubes are used, and each is filled with 10 mL 0.04% starch solution. 1 drop of Buzyme 2506 (amylase product) is added to one of these test tubes, and the test tube is shaken well. After waiting approximately 10 seconds, 0.5 mL 0.01 M I₂/KI solution is added to each test tube. In an embodiment, up to 1.0 mL 0.01 M I₂/KI solution may be added. The solution containing Buzyme 2506 shows a lighter color due to lower starch concentration.

This test uses modified starch-based product BBD 616 to represent all kinds of starch in OCC. Series dilution is necessary to obtain an accurate standard working starch solution concentration curve. Starch-iodine solution only at low concentration has good linear dosage response.

The above method is useful as a quick and simple method to determine a starch concentration. This method also works well for monitoring starch concentration change along a repulping process.

Free starch and bonded starch ratio is highly related to repulping conditions, such as time, shear force, pH, and consistency. Factors which result in higher ratio of free starch include, for example, longer repulping time, higher shear force, higher temperature, lower pH, and lower consistency.

Since absorbance of starch-iodine solutions changes with time, these solutions should be made fresh and tested immediately after being made.

Starch data may be combined with other paper mill-related data. For example, starch content may influence microbial contamination, and the starch concentration obtained by the methods herein may be used to determine MB contamination.

Advantages and benefits of the methods described herein include ease of use at a paper mill; accurate data; measurements of free starch, total starch, and percentage of fiber-bonded starch; and data useful for return-on-investment calculations. Additionally, if sample consistencies are known, the data can be offset, and starch percentage may be obtained by dry fiber weight.

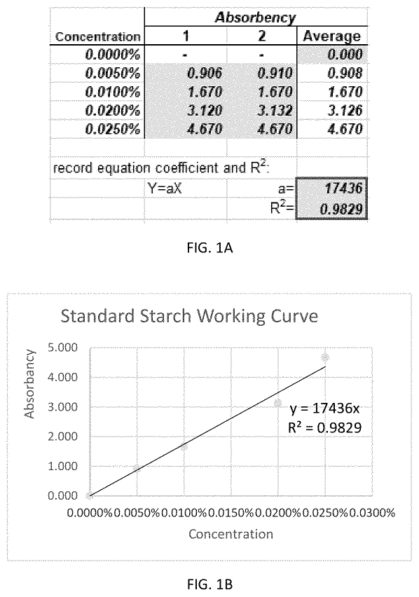

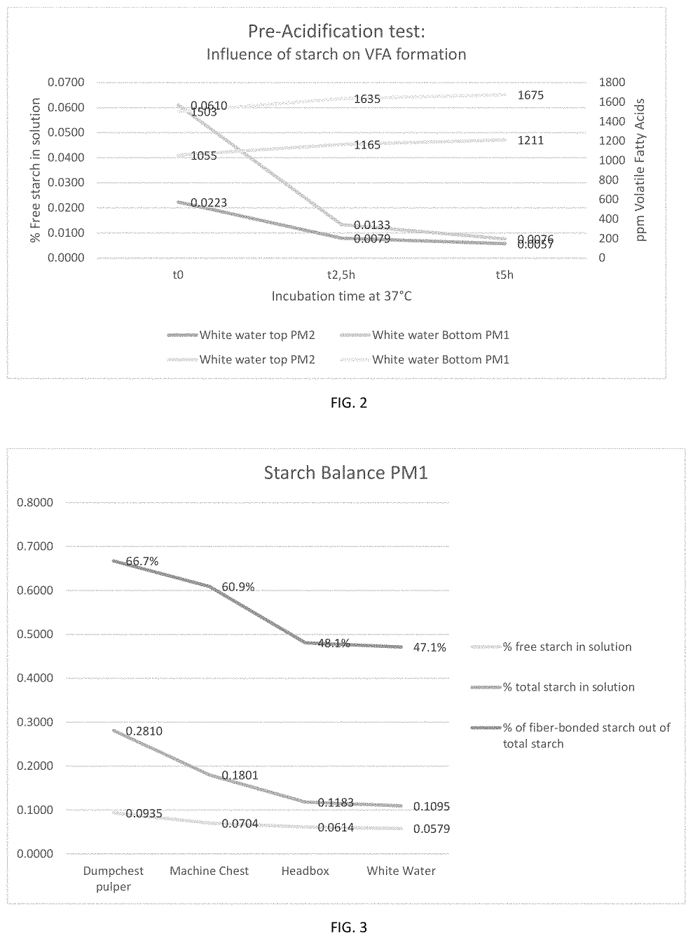

The light absorbance of iodine-starch solutions as a function of starch was measured at 620 nm with a spectrometer, and absorbance values for samples (with concentrations of 0.0000%, 0.0050%, 0.0100%, 0.0200%, and 0.0250%)—and averages thereof—prepared according to the disclosure are shown in FIG. 1A.

FIG. 1B shows a standard starch working curve (or linear correlation) prepared using the average concentration values obtained in FIG. 1A.

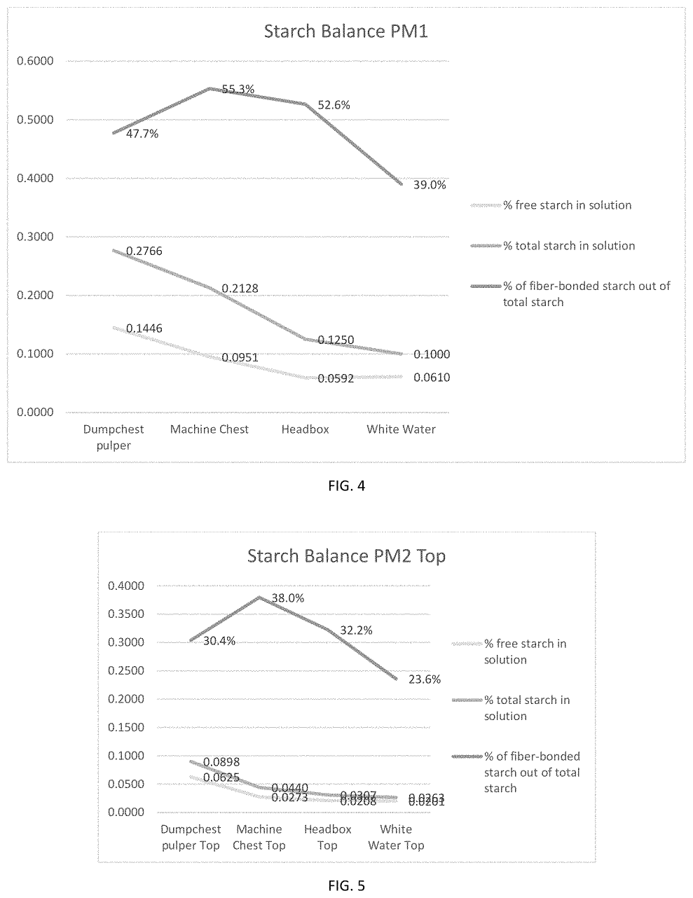

FIG. 2 graphically represents the influence of starch on volatile fatty acid formation in a pre-acidification test; and

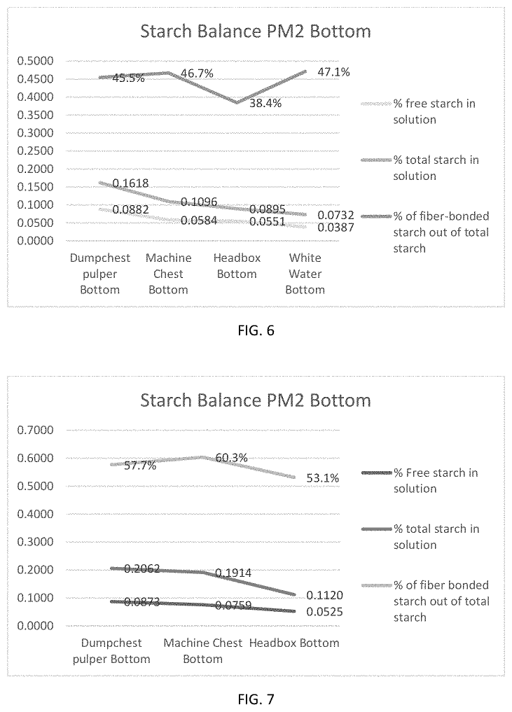

FIGS. 3-7 show, for various samples, percentage free starch in solution, percentage total starch in solution, and percentage of fiber-bonded starch out of total starch.

A method according to embodiments of the invention can be used in measurements in a laboratory, a plant or a mill, for example.

In embodiments of the invention, different particle populations in the liquid samples such as pulp suspension or filtrate may be distinguished or separated from each other.

In embodiments of the invention, the sample containing particles is mixed with an iodine solution. The iodine concentration in a sample may be selected according to an application. Examples of different iodine concentrations in a sample are given above.

In embodiments of the inventions, the sample may be separated into one or more particle populations according to a particle size before the step of adding the iodine solution. A fraction or population may comprise the dissolved liquid portion of the sample, including the dissolved starch, without particulate matter.

In embodiments of the invention, starch concentration may be measured for one or more different fractions or particle populations. A fraction or population may comprise the dissolved liquid portion of the sample, including the dissolved starch, without particulate matter.

In embodiments of the invention, the light absorbance or transmittance of the sample may be measured for two or more different fractions or particle populations, and the measured absorbance or transmittance of the samples converted into the starch concentration of the sample for each particle population by means of a predefined correlation between the starch concentration and the light absorbance or transmittance.

In embodiments of the invention, the light absorbance or transmittance of the sample may be measured for two or more different fractions or particle populations both before and after the step of adding the iodine solution, a difference between the two measured absorbance or transmittance of the sample is converted into the starch concentration of the sample for each particle population by means of the predefined correlation between the starch concentration and the light absorbance or transmittance.

In embodiments of the invention, a ratio of dissolved and absorbed starch in the sample may be determined based on the starch concentrations of different particle populations.

The method can be used for measurement of soluble starch but can also be extended to measure larger particles, such as fines, fibers, floccules and agglomerates.

The obtained concentration of dissolved and absorbed starch can be utilized for total chemistry management in paper, board and tissue processes. Typical applications may include retention, sizing, strength, deposit control and microbe control. Typical measuring locations may include wet end, broke line, pulp filtrates and long circulation.

The obtained concentration of dissolved and absorbed starch can be utilized for monitoring chemistry performance and controlling chemical dosages. Control can be manual or automatic.

It is to be understood that the embodiments of the invention disclosed are not limited to the particular structures, process steps, or materials disclosed herein, but are extended to equivalents thereof as would be recognized by those ordinarily skilled in the relevant arts.

It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention. Well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A method of analyzing starch concentration in a liquid sample, comprising
obtaining a sample from a stream of liquid;
adding iodine solution to the sample;
measuring a light absorbance or transmittance of the sample; and
converting the measured absorbance or transmittance of the sample into a starch concentration of the sample by correlating the starch concentration and the light absorbance or transmittance.

2. The method of claim 1, further comprising
separating the sample into one or more particle populations according to a particle size and/or mass before adding the iodine solution;
measuring the light absorbance or transmittance of the sample for each particle population of the sample; and
converting the measured absorbance or transmittance of the sample into the starch concentration of the sample for each particle population by means of the correlation between the starch concentration and the light absorbance or transmittance.

3. The method of claim 2, wherein the one or more particle populations comprises a population containing dissolved starch without particulate matter.

4. The method of claim 3, further comprising
determining a ratio of dissolved and absorbed starch in the sample based on the starch concentrations of the one or more particle populations.

5. The method of claim 3, wherein the one or more particle populations include one or more of colloids, fines, fibers, floccules and agglomerates.

6. The method of claim 1, wherein the liquid sample is obtained from a pulp suspension or filtrate in a paper, board or tissue process.

7. A measurement system implementing the method as claimed in claim 1.

8. The method of claim 1, wherein the measuring a light absorbance or transmittance of the sample is at a wavelength of between 600-630 nm.

9. The method of claim 8, wherein the measuring comprises measuring the light absorbance of the sample after adding the iodine solution.

10. The method of claim 8, wherein the measuring comprises measuring the light transmittance of the sample after adding the iodine solution.

11. The method of claim 8, wherein the measuring comprises measuring the light absorbance or transmittance of the sample at a wavelength of between 610-625 nm after adding the iodine solution.

12. The method of claim 8, wherein the measuring comprises measuring the light absorbance or transmittance of the sample at a wavelength of 620 nm after adding the iodine solution.

13. The method of claim 1, wherein the iodine solution comprises between 0.05-1 mL 0.01 M I₂/KI solution.

14. The method of claim 1, further comprising
measuring a first light absorbance or transmittance of the sample before the iodine solution is added and
measuring a second light absorbance or transmittance of the sample after the iodine solution is added.
